(19) United States
(12) Patent Application Publication
(10) Pub. No.: US 2023/0248977 A1
(43) Pub. Date: Aug. 10, 2023

(54) AUTOMATIC THERAPY ADJUSTMENT BASED ON INTERNAL AND EXTERNAL SENSING

(71) Applicant: Boston Scientific Neuromodulation Corporation, Valencia, CA (US)

(72) Inventors: Rosana Esteller, Santa Clarita, CA (US); Andrew James Haddock, Los Angeles, CA (US); Mahsa Malekmohammadi, Sherman Oaks, CA (US); Tianhe Zhang, Studio City, CA (US); Venugopal Allavatam, Saratoga, CA (US)

(21) Appl. No.: 18/107,341

(22) Filed: Feb. 8, 2023

Related U.S. Application Data
(60) Provisional application No. 63/308,574, filed on Feb. 10, 2022.

(51) Int. Cl. A61N 1/36; A61B 5/388; A61B 5/11

(52) U.S. Cl. CPC A61N 1/36139; A61B 5/388; A61N 1/3614; A61B 5/1101

(57) ABSTRACT

This document discusses a computer-implemented method of calibration of an implantable neurostimulation device. The method includes sensing one or more symptoms of a neurological condition of a subject using a sensor external to the neurostimulation device; delivering neurostimulation to the subject using the neurostimulation device and adjusting neurostimulation parameters based on the sensed symptom; sensing one or more neural response signals resulting from the neurostimulation using a sensor of the neurostimulation device; correlating the sensed symptom with the one or more sensed neural response signals; determining a target neural response using the correlating; and recurrently adjusting the neurostimulation parameters according to a comparison of subsequently sensed neural response signals to the target neural response signal.

### CLAIM OF PRIORITY

This application claims the benefit of U.S. Provisional Application No. 63/308,574, filed on Feb. 10, 2022, which is hereby incorporated by reference in its entirety.

### TECHNICAL FIELD

This document relates generally to medical devices and more particularly to a system for neurostimulation.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device can be used to program the implantable neurostimulator with stimulation parameters controlling the delivery of the neurostimulation energy.

In one example, the neurostimulation energy is delivered in the form of electrical neurostimulation pulses. The delivery is controlled using stimulation parameters that specify spatial (where to stimulate), temporal (when to stimulate), and informational (patterns of pulses directing the nervous system to respond as desired) aspects of a pattern of neurostimulation pulses. Many current neurostimulation systems are programmed to deliver periodic pulses with one or a few uniform waveforms continuously or in bursts. However, the human nervous systems use neural signals having much more sophisticated features to communicate various types of information, including sensations of pain, pressure, temperature, etc. The present inventor has recognized a need for improvement in the electrical neurostimulation provided by medical devices.

### SUMMARY

Electrical neurostimulation energy can be delivered in the form of electrical neurostimulation pulses to treat a neurological condition of the patient. The response of the patient to the treatment may change with time after the initial parameters of the neurostimulation are set. Changes in the response to the treatment may require subsequent visits to a clinic to reset the neurostimulation to address the changes.

Example 1 includes subject matter (such as a computer-implemented method of calibration of an implantable neurostimulation device) comprising sensing one or more symptoms of a neurological condition of a subject using one or more sensors external to the neurostimulation device, delivering neurostimulation to the subject using the neurostimulation device and adjusting neurostimulation parameters based on the sensed one or more symptoms, sensing one or more neural response signals resulting from the neurostimulation using a sensor of the neurostimulation device, correlating the one or more sensed symptoms with the one or more sensed neural response signals, determining a target neural response using the correlating, and recurrently adjusting the neurostimulation parameters according to a comparison of subsequently sensed neural response signals to the target neural response signal.

In Example 2, the subject matter of Example 1 optionally includes ending the adjusting of the neurostimulation parameters based on the one or more sensed symptoms and continuing the adjusting of the neurostimulation parameters according to the comparison of subsequently sensed neural response signals to the target neural response signal.

In Example 3, the subject matter of one or both of Examples 1 and 2 optionally includes detecting a change in at least one sensed symptom of the one or more sensed symptoms, and changing the target neural response signal based on the detected change in the at least one sensed symptom.

In Example 4, the subject matter of one or any combination of Examples 1-3 optionally includes detecting a change in at least one sensed symptom of the one or more sensed symptoms, and enabling the recurrent adjusting the neurostimulation parameters in response to the detected change in the at least one sensed symptom.

In Example 5, the subject matter of one or any combination of Examples 1-4 optionally includes sensing one or more evoked potential signals, and the target neural response signal is a target evoked potential signal.

In Example 6, the subject matter of Example 5, optionally includes sampling the one or more evoked potential signals, and generating a template of the target evoked potential signal using one or more sampled evoked potential signals.

In Example 7, the subject matter of one or any combination of Examples 1-6 optionally includes sensing the one or more neural response signals using a device separate from the neurostimulation device, generating the target neural response signal using the separate device, and sending the target neural response signal to the neurostimulation device and storing the target neural response in memory of the neurostimulation device.

In Example 8, the subject matter of one or any combination of Examples 1-7 optionally includes sensing one or more evoked resonant neural activity signals, one or more local field potential signals, or one or more stimulation artifact signals.

In Example 9, the subject matter of one or any combination of Examples 1-8 optionally includes sensing lead impedance of a lead used to deliver the neurostimulation, comparing the sensed lead impedance to a specified lead impedance range, and sending an indication associated with the sensed lead impedance to a user or process when the sensed lead impedance is outside the specified lead impedance range.

In Example 10, the subject matter of one or any combination of Examples 1-9 optionally includes adjusting the neurostimulation parameters according to the sensing of the subsequent neural response signals and according to a medication schedule of the subject.

In Example 11, the subject matter of one or any combination of Examples 1-10 optionally includes the one or more sensed symptoms including a tremor, and the one or more external sensors including a motion sensor.

In Example 12, the subject matter of one or any combination of Examples 1-11 optionally includes the one or more sensed symptoms including abnormal gait of the subject, and the one or more external sensors including a motion sensor.

In Example 13, the subject matter of one or any combination of Examples 1-12 optionally includes the neurostimulation device being an implantable pulse generator that includes the internal sensor and the one or more external sensors being wearable sensors.

In Example 14, the subject matter of one or any combination of Examples 1-13 optionally includes recording one or more signals sensed by the external sensor in response to delivering the neurostimulation.

Example 15 includes subject matter (such as a medical device system) or can optionally be combined with one or any combination of Examples 1-14 to include such subject matter, comprising an implantable neurostimulation device including a therapy circuit configured to deliver electrical neurostimulation to a subject when coupled to implantable electrodes, at least one external sensor configured to detect at least one symptom of a neurological condition of the subject, and an external device. The external device includes an external control circuit configured to receive information of the at least one detected symptom from the external sensor, and set one or more neurostimulation parameters of the neurostimulation delivered by the neurostimulation device according to the at least one detected symptom. The implantable neurostimulation device also including a sensor circuit configured to sense one or more neural response signals resulting from the neurostimulation, and an internal control circuit, operatively coupled to the therapy circuit and the sensor circuit, and configured to recurrently adjust the one or more neurostimulation parameters based on a comparison of the target neural response signal to the sensed neural response signals.

In Example 16, the subject matter of Example 15 optionally includes an external device including a neural signal sensing circuit configured to sense the one or more neural signals, signal processing circuitry configured to produce the target neural response signal, and a communication circuit configured to transfer the target neural response signal to memory of the implantable neurostimulation device.

In Example 17, the subject matter of one or both of Examples 15 and 16 optionally includes a communication circuit configured to receive a prompt from the external device, and signal processing circuitry configured to produce the target neural response signal in response to a prompt received from the external device.

Example 18 includes subject matter (such as an electronic device) or can optionally be combined with one or any combination of Examples 1-17 to include such subject matter, comprising one or more sensors configured to detect one or more symptoms of a neurological condition of a subject, a communication circuit configured to transfer information to a separate device, a control circuit operatively coupled to the one or more sensors and the communication circuit, and memory. The memory stores an application that includes instructions that when performed by the control circuit, causes the control circuit to perform operations including communicate one or more neurostimulation parameters of neurostimulation according to the detected one or more symptoms, wherein the neurostimulation is provided by a separate neurostimulation device; initiate a transfer of a target neural response signal to the neurostimulation device; and communicate a prompt to cause the neurostimulation device to recurrently adjust the one or more neurostimulation parameters to reduce a difference between a neural response signal sensed by the neurostimulation device and the target neural response signal.

In Example 19, the subject matter of Example 18 optionally includes an application including instructions that when performed by the control circuit, causes the control circuit to perform operations including transfer symptom information of the detected symptom to a separate programming device; and receive the target neural response signal from the separate device.

In Example 20, the subject matter of one or both of Examples 18 and 19 optionally includes the one or more sensors, the communication circuit, the control circuit, and the memory are included in a mobile device.

These non-limiting examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of an electrical stimulation system.

FIG. 2 is a schematic side view of an example of an electrical stimulation lead.

FIGS. 3A-3H are illustrations of different embodiments of leads with segmented electrodes.

FIG. 4 is a block diagram of portions of an example of a medical device for providing neurostimulation.

FIG. 5 is block diagram of a method of closed loop feedback control of neurostimulation therapy provided by a neurostimulation device.

FIG. 6 is a block diagram of a method of calibration of operation of a neurostimulation device.

FIGS. 7A-7B are block diagrams of examples of methods of adjusting closed loop feedback control of a neurostimulation device.

FIG. 8 is an example of a graphical user interface (GUI) screen useful for calibration of a neurostimulation device.

FIG. 9 is an example of a GUI screen useful to configure closed loop operation of a neurostimulation device.

FIGS. 10A-10B are another example of a GUI screen useful for calibration of a neurostimulation device.

FIG. 11 is another example of a GUI screen useful for calibration of a neurostimulation device.

FIG. 12 is a block diagram of a medical device system.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses devices, systems and methods for programming and delivering electrical neurostimulation to a patient or subject. Advancements in neuroscience and neurostimulation research have led to a demand for delivering complex patterns of neurostimulation energy for various types of therapies. The present system may be implemented using a combination of hardware and software designed to apply any neurostimulation (neuromodulation) therapy, including but not being limited to DBS, SCS, PNS, FES, and Vagus Nerve Stimulation (VNS) therapies.

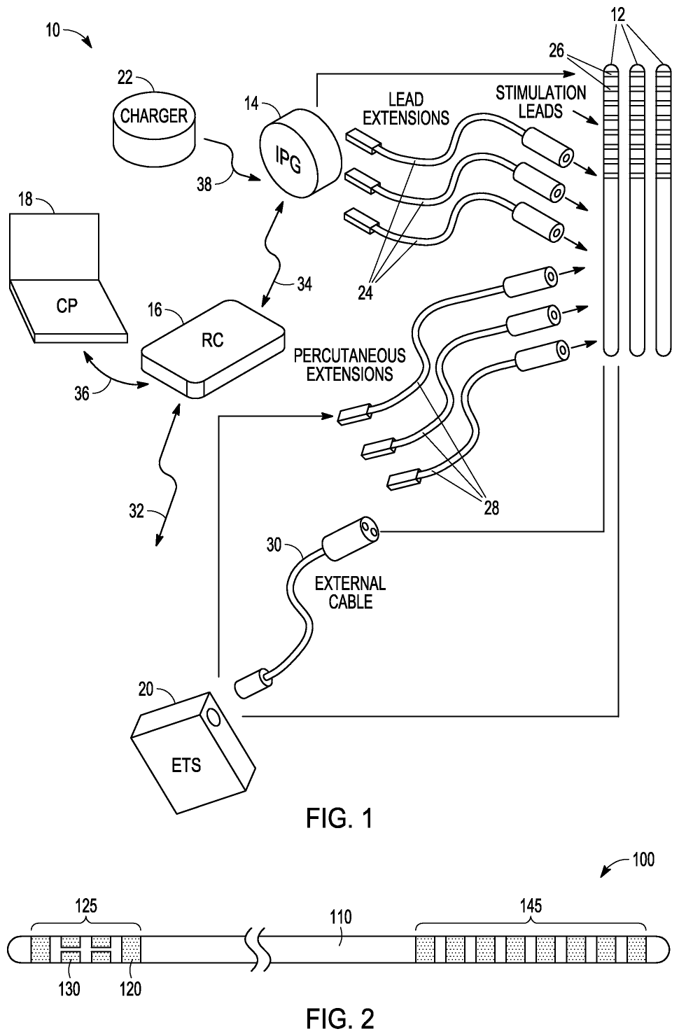

FIG. 1 is an illustration of portions of an embodiment of an electrical stimulation system 10 that includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG 14 can optionally be physically connected via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The implantable pulse generator can have multiple stimulation channels (e.g., 8 or 16) which may be independently programmable to control the magnitude of the current stimulus from each channel. The IPG 14 can have one, two, three, four, or more connector ports, for receiving the terminals of the leads 12.

The ETS 20 may also be physically connected, optionally via the percutaneous lead extensions 28 and external cable 30, to the stimulation leads 12. The ETS 20, which may have similar pulse generation circuitry as the IPG 14, can also deliver electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a wireless communications link 32. Once the IPG 14 and neurostimulation leads 12 are implanted, the RC 16 may be used to telemetrically communicate with or control the IPG 14 via communications link 34. The communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is incorporated herein by reference. Other embodiments of electrical stimulation systems can be found at U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; 8,364,278; and 8,700,178, all of which are incorporated herein by reference.

FIG. 2 is a schematic side view of an embodiment of an electrical stimulation lead. FIG. 2 illustrates a lead 110 with electrodes 125 disposed at least partially about a circumference of the lead 110 along a distal end portion of the lead and terminals 145 disposed along a proximal end portion of the lead. The lead 110 can be implanted near or within the desired portion of the body to be stimulated (e.g., the brain, spinal cord, or other body organs or tissues). In one example of operation for deep brain stimulation, access to the desired position in the brain can be accomplished by drilling a hole in the patient's skull or cranium with a cranial drill (commonly referred to as a burr), and coagulating and incising the dura mater, or brain covering. The lead 110 can be inserted into the cranium and brain tissue with the assistance of a stylet (not shown). The lead 110 can be guided to the target location within the brain using, for example, a stereotactic frame and a microdrive motor system. In some embodiments, the microdrive motor system can be fully or partially automatic. The microdrive motor system may be configured to perform one or more the following actions (alone or in combination): insert the lead 110, advance the lead 110, retract the lead 110, or rotate the lead 110.

In some embodiments, measurement devices coupled to the muscles or other tissues stimulated by the target neurons, or a unit responsive to the patient or clinician, can be coupled to the implantable pulse generator or microdrive motor system. The measurement device, user, or clinician can indicate a response by the target muscles or other tissues to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

The lead 110 for deep brain stimulation can include stimulation electrodes, recording electrodes, or both. In at least some embodiments, the lead 110 is rotatable so that the stimulation electrodes can be aligned with the target neurons after the neurons have been located using the recording electrodes. Stimulation electrodes may be disposed on the circumference of the lead 110 to stimulate the target neurons. Stimulation electrodes may be ring-shaped so that current projects from each electrode equally in every direction from the position of the electrode along a length of the lead 110. In the embodiment of FIG. 2, two of the electrodes 120 are ring electrodes 120. Ring electrodes typically do not enable stimulus current to be directed from only a limited angular range around of the lead. Segmented electrodes 130, however, can be used to direct stimulus current to a selected angular range around the lead. When segmented electrodes are used in conjunction with an implantable pulse generator that delivers constant current stimulus, current steering can be achieved to more precisely deliver the stimulus to a position around an axis of the lead (e.g., radial positioning around the axis of the lead). To achieve current steering, segmented electrodes can be utilized in addition to, or as an alternative to, ring electrodes.

The lead 100 includes a lead body 110, terminals 145, and one or more ring electrodes 120 and one or more sets of segmented electrodes 130 (or any other combination of electrodes). The lead body 110 can be formed of a biocompatible, non-conducting material such as, for example, a polymeric material. Suitable polymeric materials include, but are not limited to, silicone, polyurethane, polyurea, polyurethaneurea, polyethylene, or the like. Once implanted in the body, the lead 100 may be in contact with body tissue for extended periods of time. In at least some embodiments, the lead 100 has a cross-sectional diameter of no more than 1.5 millimeters (1.5 mm) and may be in the range of 0.5 to 1.5 mm. In at least some embodiments, the lead 100 has a length of at least 10 centimeters (10 cm) and the length of the lead 100 may be in the range of 10 to 70 cm.

The electrodes 125 can be made using a metal, alloy, conductive oxide, or any other suitable conductive biocompatible material. Examples of suitable materials include, but are not limited to, platinum, platinum iridium alloy, iridium, titanium, tungsten, palladium, palladium rhodium, or the like. Preferably, the electrodes are made of a material that is biocompatible and does not substantially corrode under expected operating conditions in the operating environment for the expected duration of use. Each of the electrodes can either be used or unused (OFF). When the electrode is used, the electrode can be used as an anode or cathode and carry anodic or cathodic current. In some instances, an electrode might be an anode for a period of time and a cathode for a period of time.

Deep brain stimulation leads and other leads may include one or more sets of segmented electrodes. Segmented electrodes may provide for superior current steering than ring electrodes because target structures in deep brain stimulation or other stimulation are not typically symmetric about the axis of the distal electrode array. Instead, a target may be located on one side of a plane running through the axis of the lead. Through the use of a radially segmented electrode array (“RSEA”), current steering can be performed not only along a length of the lead but also around a circumference of the lead. This provides precise three-dimensional targeting and delivery of the current stimulus to neural target tissue, while potentially avoiding stimulation of other tissue.

Embodiments of leads with segmented electrodes include U.S. Pat. Nos. 8,473,061; 8,571,665; and 8,792,993; U.S. Patent Application Publications Nos. 2010/0268298; 2011/0005069; 2011/0130803; 2011/0130816; 2011/0130817; 2011/0130818; 2011/0078900; 2011/0238129; 2012/0016378; 2012/0046710; 2012/0071949; 2012/0165911; 2012/0197375; 2012/0203316; 2012/0203320; 2012/0203321; 2013/0197424; 2013/0197602; 2014/0039587; 2014/0353001; 2014/0358208; 2014/0358209; 2014/0358210; 2015/0045864; 2015/0066120; 2015/0018915; 2015/0051681; 2015/0151113; and 2014/0358207; all of which are incorporated herein by reference.

Any number of segmented electrodes 130 may be disposed on the lead body 110 including, for example, anywhere from one to sixteen or more segmented electrodes 130. It will be understood that any number of segmented electrodes 130 may be disposed along the length of the lead body 110. A segmented electrode 130 typically extends only 75%, 67%, 60%, 50%, 40%, 33%, 25%, 20%, 17%, 15%, or less around the circumference of the lead.

The segmented electrodes 130 may be grouped into sets of segmented electrodes, where each set is disposed around a circumference of the lead 100 at a particular longitudinal portion of the lead 100. The lead 100 may have any number segmented electrodes 130 in a given set of segmented electrodes. The lead 100 may have one, two, three, four, five, six, seven, eight, or more segmented electrodes 130 in a given set. In at least some embodiments, each set of segmented electrodes 130 of the lead 100 contains the same number of segmented electrodes 130. The segmented electrodes 130 disposed on the lead 100 may include a different number of electrodes than at least one other set of segmented electrodes 130 disposed on the lead 100. The segmented electrodes 130 may vary in size and shape. In some embodiments, the segmented electrodes 130 are all of the same size, shape, diameter, width or area or any combination thereof. In some embodiments, the segmented electrodes 130 of each circumferential set (or even all segmented electrodes disposed on the lead 100) may be identical in size and shape.

Each set of segmented electrodes 130 may be disposed around the circumference of the lead body 110 to form a substantially cylindrical shape around the lead body 110. The spacing between individual electrodes of a given set of the segmented electrodes may be the same, or different from, the spacing between individual electrodes of another set of segmented electrodes on the lead 100. In at least some embodiments, equal spaces, gaps or cutouts are disposed between each segmented electrode 130 around the circumference of the lead body 110. In other embodiments, the spaces, gaps or cutouts between the segmented electrodes 130 may differ in size, or cutouts between segmented electrodes 130 may be uniform for a particular set of the segmented electrodes 130 or for all sets of the segmented electrodes 130. The sets of segmented electrodes 130 may be positioned in irregular or regular intervals along a length the lead body 110.

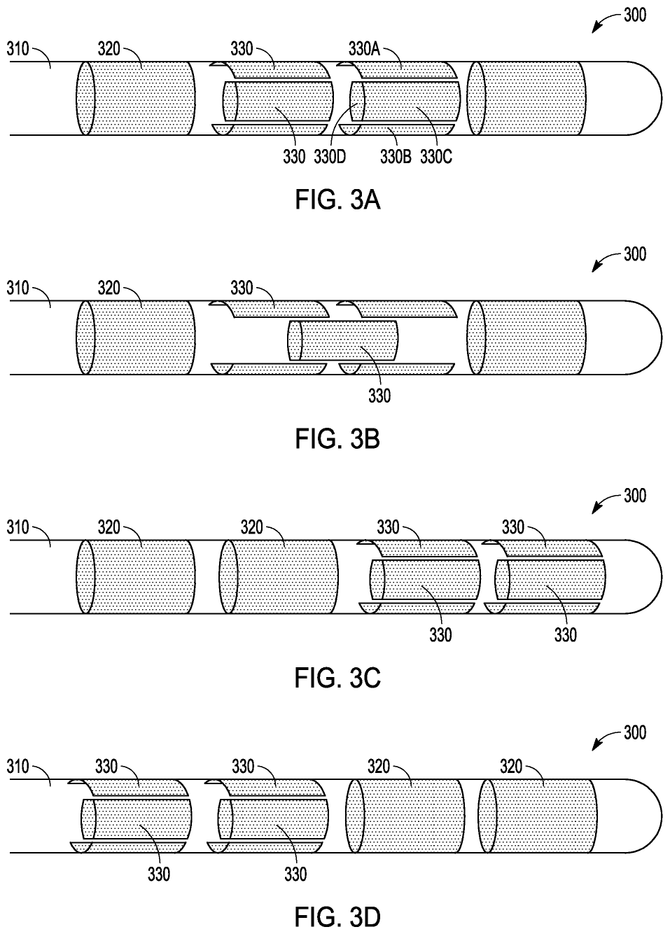

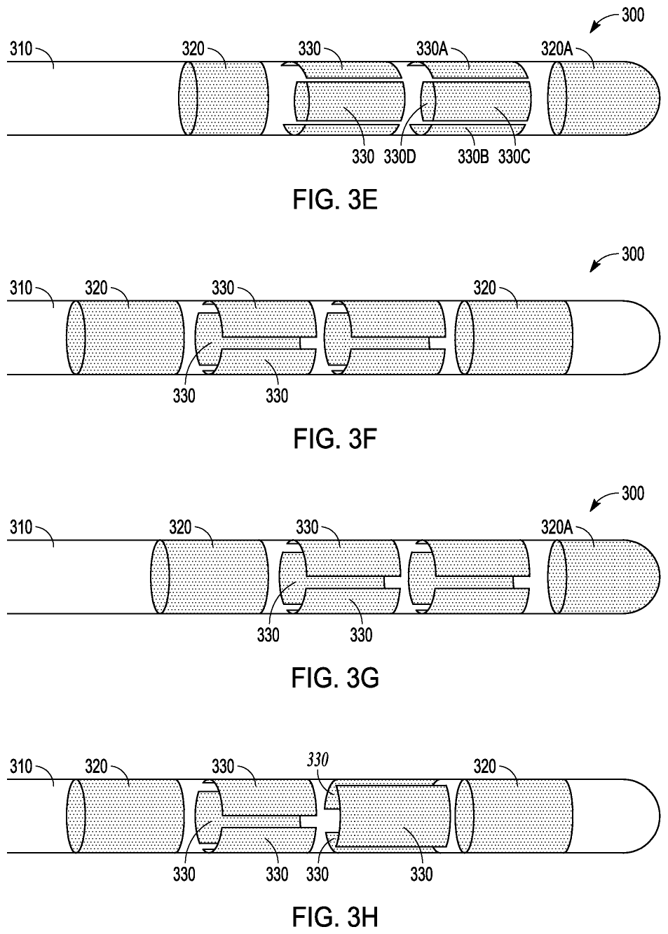

Conductor wires (not shown) that attach to the ring electrodes 120 or segmented electrodes 130 extend along the lead body 110. These conductor wires may extend through the material of the lead 100 or along one or more lumens defined by the lead 100, or both. The conductor wires couple the electrodes 120, 130 to the terminals 145. FIGS. 3A-3H are illustrations of different embodiments of leads 300 with segmented electrodes 330, optional ring electrodes 320 or tip electrodes 320a, and a lead body 310. The sets of segmented electrodes 330 each include either two (FIG. 3B), three (FIGS. 3E-3H), or four (FIGS. 3A, 3C, and 3D) or any other number of segmented electrodes including, for example, three, five, six, or more. The sets of segmented electrodes 330 can be aligned with each other (FIGS. 3A-3G) or staggered (FIG. 3H).

When the lead 100 includes both ring electrodes 120 and segmented electrodes 130, the ring electrodes 120 and the segmented electrodes 130 may be arranged in any suitable configuration. For example, when the lead 100 includes two ring electrodes 120 and two sets of segmented electrodes 130, the ring electrodes 120 can flank the two sets of segmented electrodes 130 (see e.g., FIGS. 2, 3A, and 3E-3H, ring electrodes 320 and segmented electrode 330). Alternately, the two sets of ring electrodes 120 can be disposed proximal to the two sets of segmented electrodes 130 (see e.g., FIG. 3C, ring electrodes 320 and segmented electrode 330), or the two sets of ring electrodes 120 can be disposed distal to the two sets of segmented electrodes 130 (see e.g., FIG. 3D, ring electrodes 320 and segmented electrode 330). One of the ring electrodes can be a tip electrode (see e.g., tip electrode 320a of FIGS. 3E and 3G). It will be understood that other configurations are possible as well (e.g., alternating ring and segmented electrodes, or the like).

By varying the location of the segmented electrodes 130, different coverage of the target neurons may be selected. For example, the electrode arrangement of FIG. 3C may be useful if the physician anticipates that the neural target will be closer to a distal tip of the lead body 110, while the electrode arrangement of FIG. 3D may be useful if the physician anticipates that the neural target will be closer to a proximal end of the lead body 110.

Any combination of ring electrodes 120 and segmented electrodes 130 may be disposed on the lead 100. For example, the lead may include a first ring electrode 120, two sets of segmented electrodes; each set formed of four segmented electrodes 130, and a final ring electrode 120 at the end of the lead. This configuration may simply be referred to as a 1-4-4-1 configuration (FIGS. 3A and 3E, ring electrodes 320 and segmented electrode 330). It may be useful to refer to the electrodes with this shorthand notation. Thus, the embodiment of FIG. 3C may be referred to as a 1-1-4-4 configuration, while the embodiment of FIG. 3D may be referred to as a 4-4-1-1 configuration. The embodiments of FIGS. 3F, 3G, and 3H can be referred to as a 1-3-3-1 configuration. Other electrode configurations include, for example, a 2-2-2-2 configuration, where four sets of segmented electrodes are disposed on the lead, and a 4-4 configuration, where two sets of segmented electrodes, each having four segmented electrodes 130 are disposed on the lead. The 1-3-3-1 electrode configuration of FIGS. 3F, 3G, and 3H has two sets of segmented electrodes, each set containing three electrodes disposed around the circumference of the lead, flanked by two ring electrodes (FIGS. 3F and 3H) or a ring electrode and a tip electrode (FIG. 3G). In some embodiments, the lead includes 16 electrodes. Possible configurations for a 16-electrode lead include, but are not limited to 4-4-4-4; 8-8; 3-3-3-3-3-1 (and all rearrangements of this configuration); and 2-2-2-2-2-2-2-2.

Any other suitable arrangements of segmented and/or ring electrodes can be used including, but not limited to, those disclosed in U.S. Provisional Patent Application Ser. No. 62/113,291 and U.S. Patent Applications Publication Nos. 2012/0197375 and 2015/0045864, all of which are incorporated herein by reference. As an example, arrangements in which segmented electrodes are arranged helically with respect to each other. One embodiment includes a double helix.

One or more electrical stimulation leads can be implanted in the body of a patient (for example, in the brain or spinal cord of the patient) and used to stimulate surrounding tissue. The lead(s) are coupled to the implantable pulse generator (such as IPG 14 in FIG. 1).

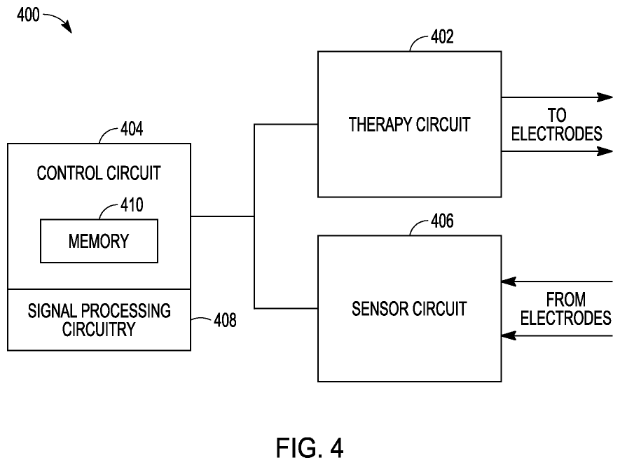

FIG. 4 is a block diagram of portions of an embodiment of a neurostimulation device 400 for providing neurostimulation. The neurostimulation device 400 may be an IPG 14. The neurostimulation device 400 includes a therapy circuit 402, a control circuit 404, and a sensor circuit 406. The therapy circuit 402 can be operatively coupled to stimulation electrodes such as any of the electrodes described herein and the therapy circuit 402 provides or delivers electrical neurostimulation energy to the electrodes. The control circuit 404 can include a processor such as a microprocessor, a digital signal processor, application specific integrated circuit (ASIC), or other type of processor, interpreting or executing instructions in software modules or firmware modules. The control circuit 404 can include other circuits or sub-circuits to perform the functions described. These circuits may include software, hardware, firmware or any combination thereof. Multiple functions can be performed in one or more of the circuits or sub-circuits as desired.

The neurostimulation device 400 includes a sensor circuit 406. An example of the sensor circuit 406 includes one or more sense amplifiers coupled to recording electrodes to sense internal neural signals of the patient. The neurostimulation device 400 can include signal processing circuitry 408 that can be integral to the control circuit 404 or separate from the control circuit 404. The signal processing circuitry 408 can include a process running on a processor to perform signal analysis or other signal processing on the neural signals sensed using the sensor circuit 406.

After implantation, a clinician will program the neurostimulation device 400 using a CP 18, remote control, or other programming device. The programmed neurostimulation device 400 can be used to treat a neurological condition of the patient, such as Parkinson's Disease, Tremor, Epilepsia, Alzheimer's Disease, other Dementias, Stroke, Multiple Sclerosis, Amyotrophic Lateral Sclerosis (ALS), Autism, brain injury, brain tumor, migraine or other pain or headache condition, and any neurological syndromes that are congenic, degenerative, or acquired.

The device-based treatment for the neurological condition of the patient would be improved through automatic therapy adjustments without the need of additional clinician programming. Sensing by the device would determine if the current treatment was meeting or not meeting a treatment target. The neurostimulation device would apply automatic feedback to adjust one or more parameters of the neurostimulation to bring the condition of the patient closer to the treatment target.

The human nervous system produces a neural response to neurostimulation received via sensory receptors or directly into any part of the network of neural elements that forms the nervous system. These neural responses are known as evoked potentials. Evoked potential (EP) signals can be sensed by the neurostimulation device 400, such as by using sense amplifiers of the device coupled to recording electrodes for example. A repetitive stimulus can be applied to the nervous system and the electrically sensed evoked potential signals can be filtered (e.g., by averaging) to detect presence of evoked potentials. An EP response can be used by the device as the target response. The neurostimulation device 400 can sense a current evoked potential signal and compare the sensed evoked potential signal to the target evoked potential signal. The neurostimulation device adjusts the neurostimulation parameters to reduce the difference between the sensed evoked potential signal and the target evoked potential signal.

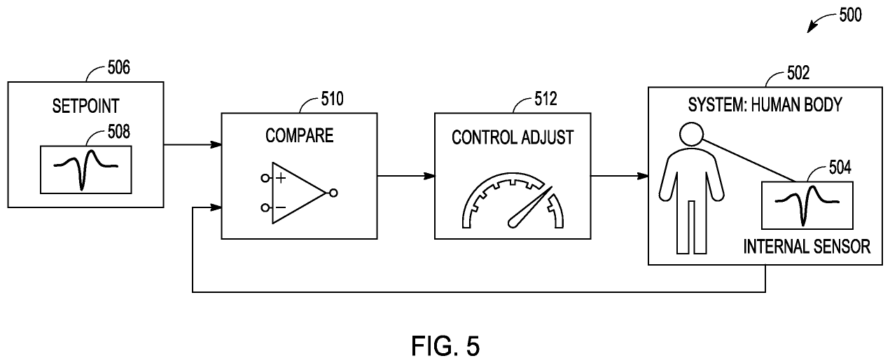

FIG. 5 is block diagram of a method 500 of closed loop feedback control of neurostimulation therapy provided by a neurostimulation device, such as the neurostimulation device of FIG. 4 for example. At block 502, the neurostimulation device provides neurostimulation therapy (e.g., using stimulation electrodes) to a patient or other subject and senses an internal neural response signal 504 using an internal sensor of the neurostimulation device. The internal sensor may include a sense amplifier circuit that is coupled to one or more recording electrodes.

The neural response signal 504 may be an EP signal, an evoked resonant neural activity (ERNA) signal also called a DLEP (DBS Local Evoked Potential), or other neural response signal spontaneously present or present as a result of the stimulation. The neural response signal may be detected in the time domain or in the frequency domain. In some examples, the neural response signal 504 may be a local field potential (LFP) signal. The neurostimulation device includes memory (e.g., memory 410 in FIG. 4) that can store a target neural response signal and features extracted from the neural response signal using the signal processing circuit (408). At block 506, the stored target neural response signal 508 or a signal feature is used to determine the Setpoint in block 506, which is used for the setpoint of the closed loop feedback control of the neurostimulation device.

At block 510, the sensed internal neural response signal 504 or extracted signal feature is compared to the target neural response signal 508 or target signal feature by the neuro stimulation device. The neurostimulation device may include signal processing circuitry to compare the internally sensed neural response signal 504 and the target neural response signal 508 or compare the feature extracted from the neural response signal sensed internally with the setpoint feature. The difference between the two signals or signal features is the feedback used to adjust the therapy provided by the device. The signal processing circuitry may calculate how close a feature of the internally sensed neural response signal 504 is to a feature of the target neural response signal 508. Alternatively, the signal processing circuitry can calculate a correlation score or a similarity metric between the signals. To do this, the signal processing circuitry may select an alignment feature in the sensed internal neural response signal 504. The signal processing circuitry may then determine a score or coefficient for how well the sensed internal neural response signal 504 correlates to the target neural response signal 508.

At block 512, the neurostimulation device adjusts one or more neurostimulation parameters when the correlation score or similarity metric is below a threshold, indicating that the sensed and target signals are too dissimilar; or alternatively, the neurostimulator device adjusts one or more stimulation parameters when the extracted feature(s) from the internally sensed neural response is very different than the setpoint feature. The neurostimulation device changes one or more neurostimulation parameters to move the morphology of the sensed neural response signal toward the morphology of the target neural response signal or the setpoint target response; or to move the morphology of an extracted feature of the sensed neural response signal closer to a feature of the setpoint target response. This restores the neurostimulation therapy to its original efficacy. Some examples of the neurostimulation parameter or parameters that may be changed include the amplitude or pulse width of neurostimulation energy pulses, charge per time unit, charge per phase, pulse frequency, and the electrodes or the electrode segmentations used to provide the neurostimulation. Other parameters may be more complex parameters, such as parameters that relate to a pattern of neurostimulation pulses provided as the neurostimulation. These patterns can include burst pulse patterns and the neurostimulation parameters can include the frequency of the pulses within a burst or the time between bursts. Other parameters can be related to one or more of pulse amplitude modulation, pulse width modulation, or pulse rate modulation of the pulse pattern, that will include modulation depth, modulation frequency, or other parameters depending on the modulation function that can be as basic as a sinewave, exponential, or can be a random sequence.

By using the target neural response signal as the setpoint, the neurostimulation device provides closed loop feedback control of the neurostimulation therapy. The feedback control can be implemented using any of proportional-integral-derivative (PID) control, a PID with thresholds, a PID with a dead-band, a lookup table, a neural network, a support vector machine (SVM), linear mean squares model, a linear regression model, a Kalman control, simple on/off control, and threshold control.

Other non-neural responses can be used as feedback. Some examples include electrical impedance of the stimulation leads and signal artifacts caused by the neurostimulation itself. Lead impedance is an indication of the coupling of the electrodes to the neural tissue. Stimulation signal artifacts can be used as an indicator of the stimulation effect or the coupling to the neural tissue, or as an indirect proxy indicator of the evoked potential response. To implement the closed loop feedback, the neurostimulation device provides neurostimulation therapy to the patient and senses a non-neural response using an internal sensor of the neurostimulation device.

A challenge with the closed loop feedback technique of FIG. 5 is calibration. The target neural response signal 508 is likely unique for a particular patient. Additionally, there may be multiple different target signals for a single patient depending on their brain state. For example, the optimal target response signal for a patient while they are sleeping may be different from the optimal target response signal when the patient is awake. The setpoint should be a target that reduces symptoms of the neurological condition of the patient while minimizing side effects, or more generally the setpoint should be a target that maintains a desired brain state or neural state. The effect of the neurostimulation therapy on one or more of the symptoms of the patient can be used to find the setpoint.

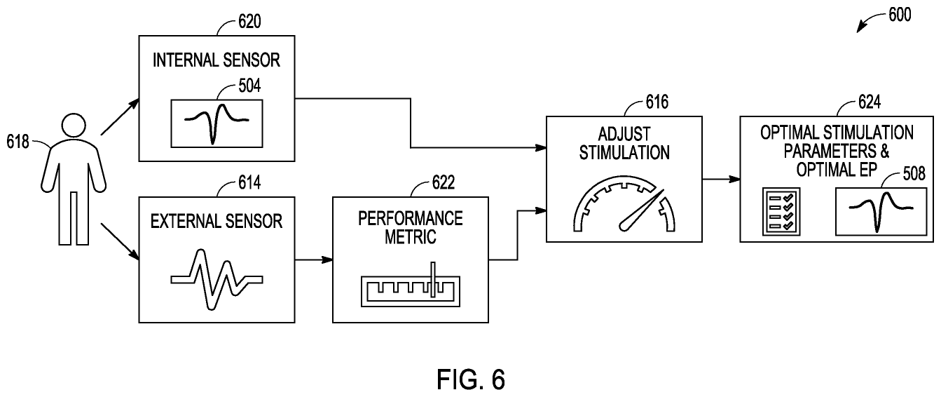

FIG. 6 is a block diagram of a method 600 of calibration of a neurostimulation device, such as the neurostimulation device of FIG. 4. At block 614, a symptom of the neurological condition of the patient is sensed using a sensor external to the neurostimulation device. For example, the symptom may be tremor of the patient and the external sensor is a motion sensor (e.g., an accelerometer, or gyroscope) that is worn by the patient (e.g., on a finger or other location). Other example symptoms include bradykinesia, rigidity in movement of the patient, or gait and balance disorders. The external sensor may be a wearable smart device (e.g., a smartwatch or health tracker) to detect the symptom or symptoms. The sensor external to the neurostimulation device is used to calibrate the stimulation provided by the device. In certain examples, the calibrating sensor is an accelerometer internal to the neural stimulation device that is used to detect a symptom related to patient mobility.

In some examples, the external sensor may be included in a smartphone or tablet computer. The symptom may be slurred speech detected using the microphone of the smartphone or tablet. The symptom may be a movement symptom detected while the patient is carrying the smartphone (e.g., detected while the patient is walking with the smartphone). The symptom may be a glucose level of the patient detected from an eye scan using the camera of the smartphone or tablet computer. The symptom may be detected by having the patient perform a task using the smartphone or tablet computer. A tablet computer may prompt the patient to re-draw a line on the touchscreen of the tablet computer and the symptom may be the patient's inability to re-draw the line. The tablet computer may extract kinematics of hand movement when the patient re-draws the line and may extract information about of the type of hand tremor detected (e.g., whether the detected tremor is an action tremor). In some examples, the symptom may be micrographia (a handwriting condition) and the tablet computer may be detected changes in the handwriting of the patient.

Other external devices can be used to detect the symptom of the patient. For example, an electrocardiograph (ECG) can be obtained for the patient and can be analyzed for arrhythmias or ECG signal morphology attributes. In certain examples, temperature of the patient outside of a temperature range may be the symptom. In certain examples, the external sensor is a chemical sensor that detects a physiological change that can impact neural responses of the patient.

At block 616, neurostimulation is delivered to the patient using the implantable neurostimulation device. The neurostimulation is adjusted by a clinician 618 to improve the symptom of the subject. For example, the clinician 618 may adjust parameters of neurostimulation provided by an IPG 14 using a CP 18, or using an ETS 20 and afterward programming an IPG 14 with the parameters. The adjustment is made to one or more neurostimulation parameters (e.g., one or more of pulse amplitude, pulse width, pulse burst pattern, etc.) to minimize or otherwise improve the symptom or symptoms of the neurological condition.

While the symptoms of the patient are being monitored and the neurostimulation is provided to the patient, at block 620 one or more neural response signals 504 (e.g., an EP signal) are sensed using a sensor of the neurostimulation device. At block 622, a performance metric is used to gauge the efficacy of the neurostimulation in improving the patient's condition. The performance metric may be a reduction in tremor, an improvement in gait, a reduction in rigidity of movement, an improvement in bradykinesia, an improvement in patient speech, an improvement in the performance of a particular task performed by the patient, etc. The status of the symptom or symptoms of the subject are correlated with the sensed neural response signals 504 or features extracted from the signals. The correlation identifies the internal neural response signals associated with improved condition of the patient as indicated by the performance metric.

At block 624, the optimal neurostimulation parameters and the optimal neural response signal are determined. The optimal neurostimulation parameters are those that produce the best performance metrics. The optimal neural response signal is used as the target neural response signal 508 in the closed loop feedback operation of the neurostimulation device as in the example of FIG. 5.

To assess neural response signals to determine a target response signal, one or more features of the neural response signal are identified and optimal values of the features are defined. Some examples include the height of any peak in the signal (e.g., amplitude of a first negative (N1) peak, the difference of peak-to-peak height between any two peaks in the signal (e.g., the difference of height of the N1 peak and height of the second positive (P2) peak), the ratio of any two peak heights in the signal (e.g., N1/P2), the width of any peak in the signal (e.g., the full-width at half-maximum of the peak), an area or energy under (or over) any peak in the signal, a peak shape selectivity metric (e.g., the time from stimulus center of any peak divided by full-width of the peak), a ratio of a selectivity metric for different peaks, the length of any portion of the curve of the signal (e.g., the length of the curve from the first positive (P1) peak to the second negative (N2) peak, any time duration defining the duration of at least a portion of the signal (e.g., the time from peak P1 to peak N2), the full-width of the signal at half-maximum, two-thirds maximum, or full maximum for any peak, or the ratio of any of these widths. Other examples include the rate of variation of any of these previous features, (e.g., how the feature or features vary with time), or any mathematical combination or function of any of these features. Also features extracting frequency domain components can be used, like to estimate changes in LFPs elicited by the stimulation. In some examples, a multidimensional cluster of the above features are included in an optimal feature set.

In some examples, neural response signals are assessed using the time delay from the time neurostimulation energy is delivered to the time of sensing of a neural response signal evoked by the neurostimulation. The time delay is indicative of the neural conduction speed of the evoked response, such as one or more of an evoked potential (EP), evoked resonant neural activity (ERNA), and evoked compound action potential (ECAP), which can be different in different types of neural tissues. In some examples, neural response signals are assessed using the conduction speed of the neural response signal, which can be determined by sensing the neural response signal as it moves past different sensing electrodes.

When adjustment of the neurostimulation parameters based on the sensed symptom ends, the implantable neurostimulation device is prompted (e.g., with a CP 18) to enter the closed feedback operation in which the implantable neurostimulation device adjusts the neurostimulation parameters based on subsequently sensed neural response signals and the setpoint for the feedback. The implantable neurostimulation device may recurrently and automatically adjust the neurostimulation parameters according to a comparison of the subsequently sensed neural response signals 504 and the target neural response signal 508 to minimize the difference between subsequent neural response signals and the target neural response signal. The comparison may include determining cross-correlation or cross-coherence of the shape of the sensed neural response signal 504 with the shape of the target neural response signal 508.

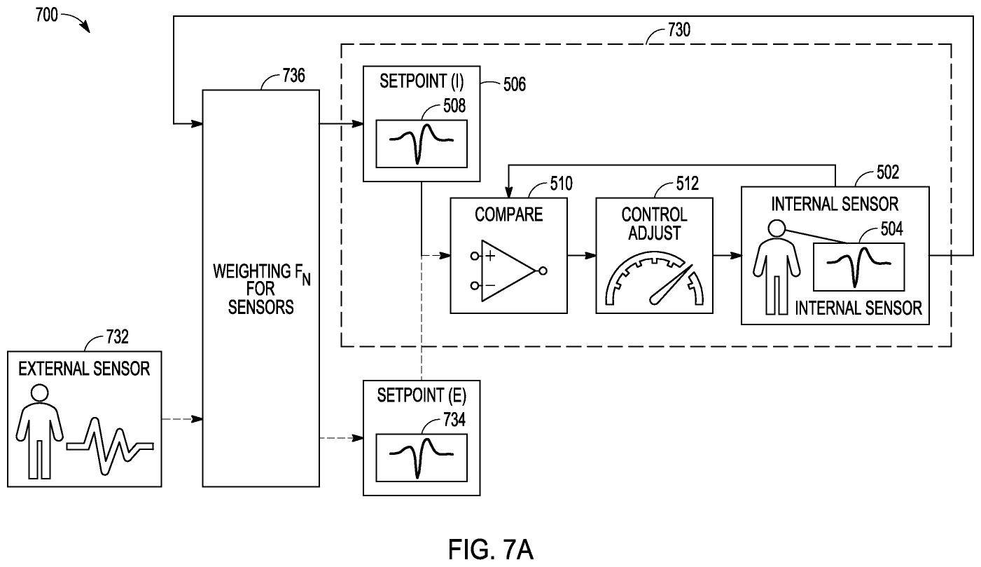

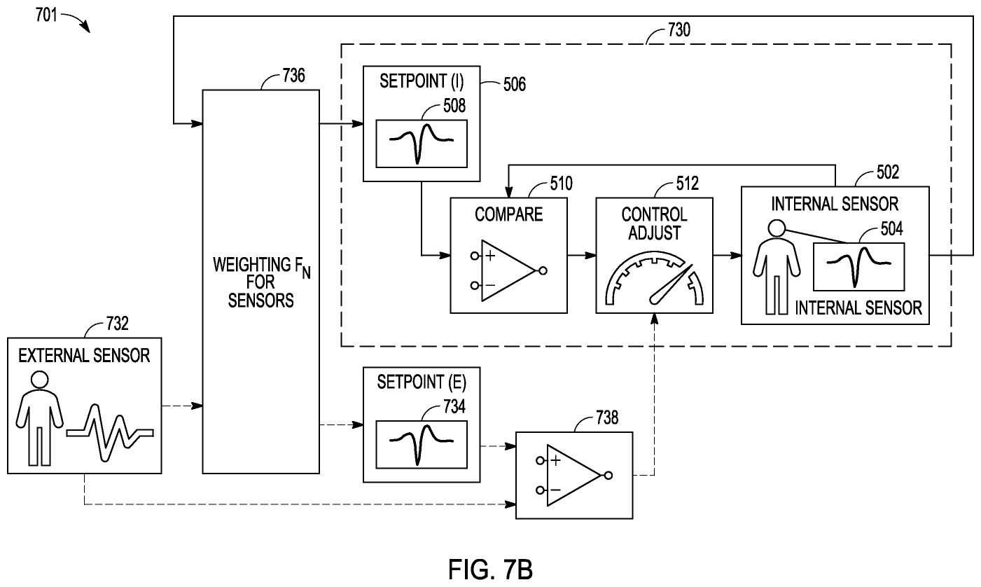

FIG. 7A is block diagram of a method 700 of adjusting the closed loop feedback control of the neurostimulation device. The closed loop feedback control portion 730 of FIG. 7A is similar to the example of FIG. 5. The neurostimulation device recurrently and automatically adjusts the neurostimulation parameters to minimize the difference between sensed neural response signals 504 or signal features and the target neural response signal 508 or signal features.

At block 732 the method 700 of FIG. 7A also uses the external sensor as input to the closed loop feedback control of the neurostimulation device. The external sensor may be any of the sensors external to the neurostimulation device that are described herein. The output of the external sensor can be used for multiple functions. In some examples, the symptom or symptoms that the neurostimulation is to address may not be constantly experienced by the patient. The output of the external sensor can indicate that the symptom is occurring and can trigger or otherwise enable the closed loop feedback control 730 of the neurostimulation device to treat the symptom. In some examples, the recurrence of the symptoms may be related to medication of the patient. For instance, the symptoms may occur at the end of a medication cycle. The closed loop feedback control can be enabled according to a medication schedule of the patient. In another example, the external sensor may indicate a change in the symptom of the patient. If a change in the symptom monitored is detected, an alert may be generated that the automatic feedback control should be recalibrated, which may involve remapping the target neural response signal 508 or Setpoint.

In some examples, the output of the external sensor is included as an input to the closed loop feedback control 730. At block 734, a setpoint is generated for the symptom and the neurostimulation is adjusted based on the internal setpoint (Setpoint (I)) and based on meeting the setpoint (Setpoint (E)) for the symptom monitored using the external sensor. In some examples, the setpoint for the symptom includes a performance metric and the neurostimulation device adjusts the neurostimulation to reduce a difference between a current value of the performance metric and the target performance metric. In some examples, the setpoint for the symptom is a signal output from the external sensor and the neurostimulation device adjusts the neurostimulation to minimize the difference between a current signal output from the external sensor and the target sensor signal.

At block 736, the outputs of the internal sensor and the external sensor can optionally be weighted as they are applied to the feedback control of the neurostimulation device. In an example, the weighting function (Fe) may weight the output of the external sensor greater than the internal sensor because it may more directly correspond to real-time symptom levels as assessed by standardized metrics (e.g., the Unified Parkinson's Disease Rate Scale (UPDRS) or The Essential Tremor Rating Assessment Scale (TETRAS)). The monitoring by the external sensor may be obtained over a different time frame than the internal sensor. In an example, the internal sensor may sense evoked potential signals when the neurostimulation is provided to the patient. In contrast, the external sensor may be a motion sensor used to monitor the gait of the patient and may only be enabled when the patient is walking or other relevant normal movements and state changes (e.g., stand vs walk vs turn, etc.).

Of note in the scheme shown in FIG. 7A, is a second feedback loop as the sensed neural response signal 504 from block 502 is input back to the system and is used at block 736 to adjust the weights from the sensors and adjust the internal Setpoint. This makes the system more flexible to operate for patient specific conditions and for likely semi-permanent neural shifts across all brain/neural states. The comparison block 510 can operate by appropriately weighting the setpoints (external and internal) and combining them into one, or by establishing two independent comparisons or each setpoint an conducting parameter adjustments accordingly.

FIG. 7B is a block diagram of another method 701 of adjusting the closed loop feedback control of the neurostimulation device. The components and blocks outside of the dashed line box may all be external and not included in an implantable neurostimulation device. In the example of FIG. 7B, there are two comparison blocks, comparison block 510 internal into the implantable neurostimulator, and an external comparison block 738 to compare the output of the external sensor with an external setpoint 734. These external and internal setpoints are compared with the actual external and internal sensed signal or signal features, and the corresponding difference is sent to the control adjust block 512 into the neurostimulation device to make the appropriate stimulation parameter adjustments. In the case of the external setpoint 734 and external comparison block 738, these functional blocks can be implemented in the cloud through appropriate software that can update the control adjust block 512 in the neurostimulation device via an application (or “app”) residing in a mobile device located with the patient.

In general, the external closed loop control that is based on external sensors do not necessarily operate in “real time” and can afford greater delays. For example, if a medication schedule is used as a control input, the elapsed time since the last medication was given is used as the external “sensing” variable. The setpoint can be for example 6 hours. After 6 hours, the medication effect starts wearing off, and the neurostimulator device may start compensating the reduced effect of the medication by appropriately adjusting one or more stimulation settings. The external setpoint can be also treated as a look up table, or as a regression function that indicates adjustment based on the time elapsed since the last medication. In an example, the patient may indicate the time of medication using the application of the patient mobile device (e.g., by clicking a user interface), and the elapsed time starts being measured from the patient indication. In another example, the application of the patient mobile device presents a trace drawing the patient rewrites in the mobile device for the external sensing to determine tremor control and establish a feature of sensed tremor to be compared against the external setpoint feature. It is to be noted that there may be multiple external sensors controlling different variables that affect the clinical symptoms and side effects of the patient, and there may be an external algorithm (e.g., running in the cloud or on the patient mobile device) that is a patient-specific algorithm that can learn from the response of the patient over time and as the external and internal sensors cumulate more information.

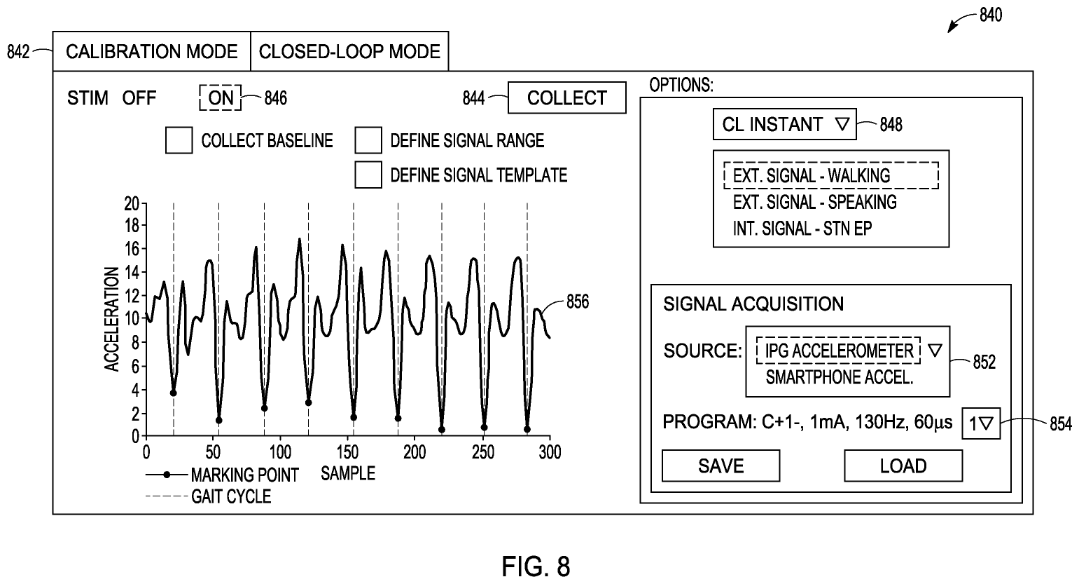

FIG. 8 is an example of a graphical user interface (GUI) screen useful for calibration of a neurostimulation device. The neurostimulation device may be an IPG 14, and the example GUI screen 840 may be presented on a display of a CP 18. The highlighted tab 842 indicates that the neurostimulation device is in calibration mode. The calibration is being performed based on gait of the patient. The GUI screen 840 shows that the signal is being collected 844 with the neurostimulation ON 846. Dropdown menu 848 indicates that the closed loop (CL) calibration is being done using walking of the patient. Other options shown in the example dropdown menu 848 include collecting a signal related to patient speech or collecting a stimulation EP signal. The signals are included in the “CL Instant” menu for “instantaneous” signals, such as walking (Ext. Signal—Walking), speech (Ext. Signal—Speaking), or acute signals such as stimulation evoked response (Int. Signal—STN EP). A different dropdown menu may be provided that includes options for chronic signals that change over a different timeframe than the instantaneous signals, such as lead impedance.

The GUI screen 840 also shows a Signal Acquisition Menu 850. The example shows options 852 for collecting the signal related to gait of the patient. The options include the accelerometer of the neurostimulation device and the accelerometer of the patient's smartphone. The Signal Acquisition Menu 850 also shows the neurostimulation program 854 used during the calibration. The example shows values for neurostimulation parameters current pulse amplitude, pulse frequency, and pulse width. The Signal Acquisition Menu 850 also shows options for saving the stimulation program or loading the program.

The calibration can be performed by having the patient walk back and forth across the room at the clinic or at a location remote from the clinic (e.g., the patient's home). The GUI displays the collected accelerometer signal 856. The spikes in the signal waveform represent points at which the patient turns around while walking. Intermittent spikes and otherwise generally low acceleration represents steady walking, while a highly variable “jittered” signal may indicate abnormal gait. Broadly speaking, features indicating abnormal gait (e.g., Parkinson's Disease gait) may be more complex than just irregular (jittered) gait. Features of abnormal gait may also include asymmetric/reduced arm swing, low speed/increased cadence, small step size, hesitation, festination, and foot shuffling without forward progression. The example GUI screen 840 provides the option to select a signal range 858. The “signal range” for a given closed loop signal represents the expected amplitude range, statistical variance, noise floor, or other range characteristic of an acquired calibration signal. The signal range may be used later to guide and calibrate therapy (e.g., to deliver neurostimulation therapy to maintain an input signal within a specified signal range).

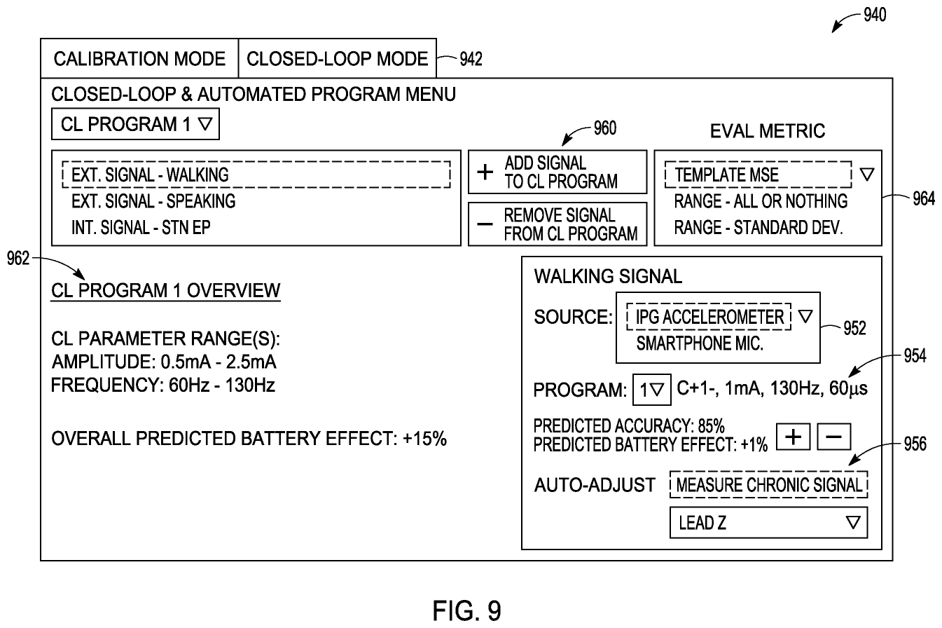

FIG. 9 is another example of a GUI screen 940. The GUI screen 940 may be presented using a display of a CP 18 to program the closed loop operation of a neurostimulation device, such as an IPG 14. The highlighted tab 942 indicates that the neurostimulation device is in closed-loop mode and the closed loop GUI screen 940 is being shown. The signal source 952 and neurostimulation program 954 are repeated from the calibration GUI screen 840. Selection windows 960 or buttons can be used to add or subtract signals from the CL Program. The GUI screen 940 also includes an overview 962 of the CL program. The overview can include parameters of the neurostimulation and the predicted effect on battery drain of operating the neurostimulation device with closed loop operation.

Evaluation Metric dropdown menu 964 selects an evaluation method with which the matching of the response to the target will be evaluated. Some examples of evaluation metrics include mean squared error (MSE) (e.g., does the MSE between the sensed response signal and the target response signal exceed a certain value or cutoff?), mean squared error of area under the curve (AUC), (e.g., does the MSE between the AUC of the sensed response signal and the AUC of the target response signal exceed a certain value of cutoff?), threshold (e.g., does the sensed response signal deviate from the target response signal by more than specified threshold difference), standard deviation in the signal range of the sensed response signal (e.g., does the sensed response signal exceed a specified standard deviation of range?), and interquartile range (e.g., does the sensed signal exceed a specified number of interquartiles of range?).

Further examples of evaluation metrics include metrics related to the power spectrums of the sensed response signal and the target response signal, and the evaluation compares power in the sensed signal to power in the target signal. The signal processing circuitry of the neural stimulation device may identify and compare metrics such as Parseval's power, peak heights, peak centers, differences over specific frequency bands, etc. In some examples, the signal processing circuitry of the neural stimulation device analyzes the evaluation metric or metrics over a windowed range. In certain examples, the signal processing circuitry of the neural stimulation device averages the response signal over the defined window and compares the averaged signal to the target signal.

If the sensed response signal deviates from the target response signal or the target response signal feature by more than a specified threshold of the evaluation metric may trigger an auto-adjustment of the neurostimulation by the closed loop algorithm. In some examples, a supplementary measurement of a second signal, (e.g., a chronic signal such as lead impedance) is used to verify that a deviation event occurred. The GUI screen 940 may include a Measure Chronic Signal menu 966 to select the chronic signal measured.

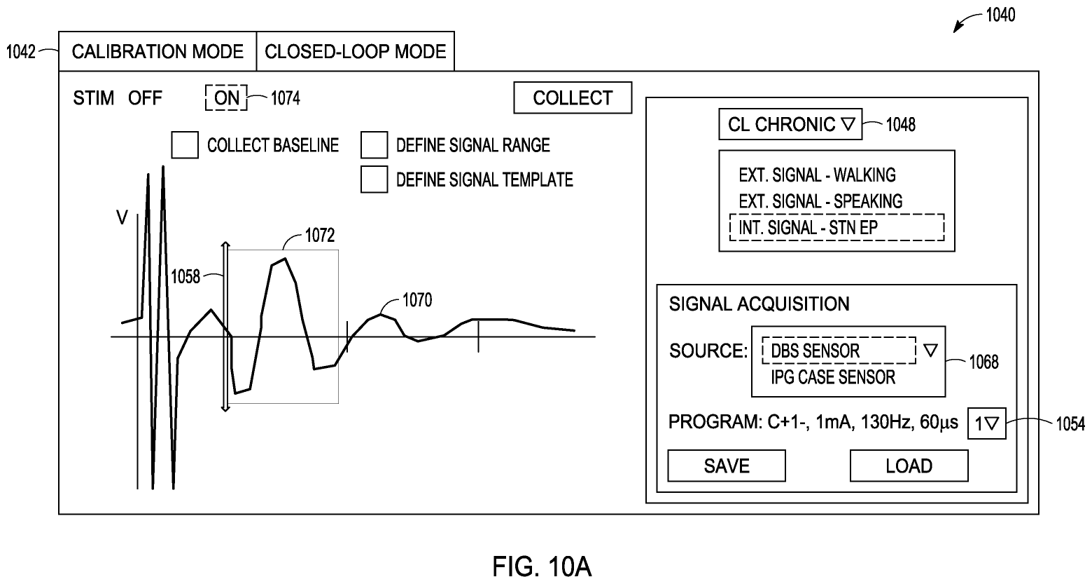

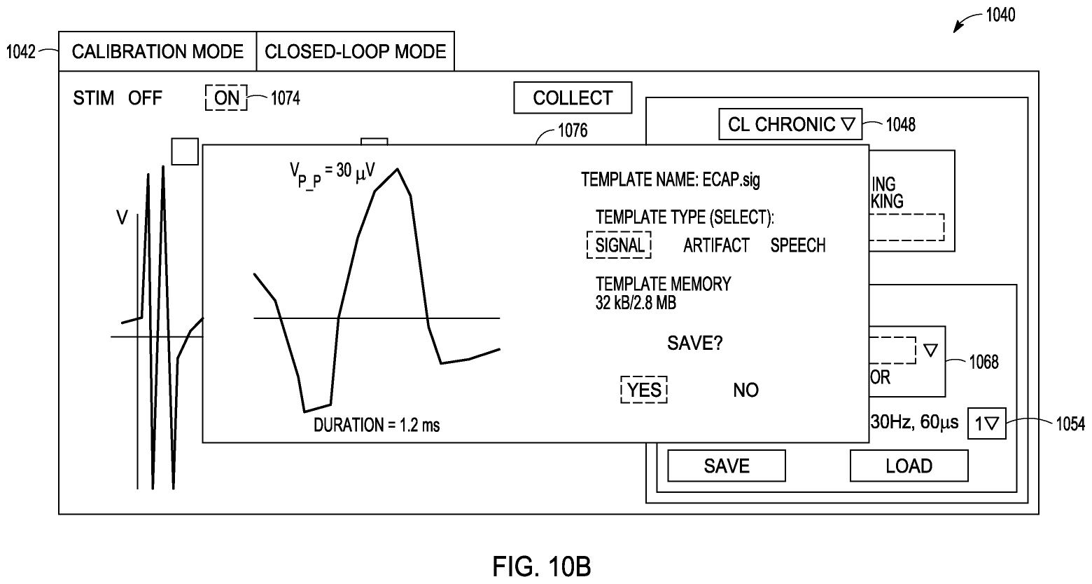

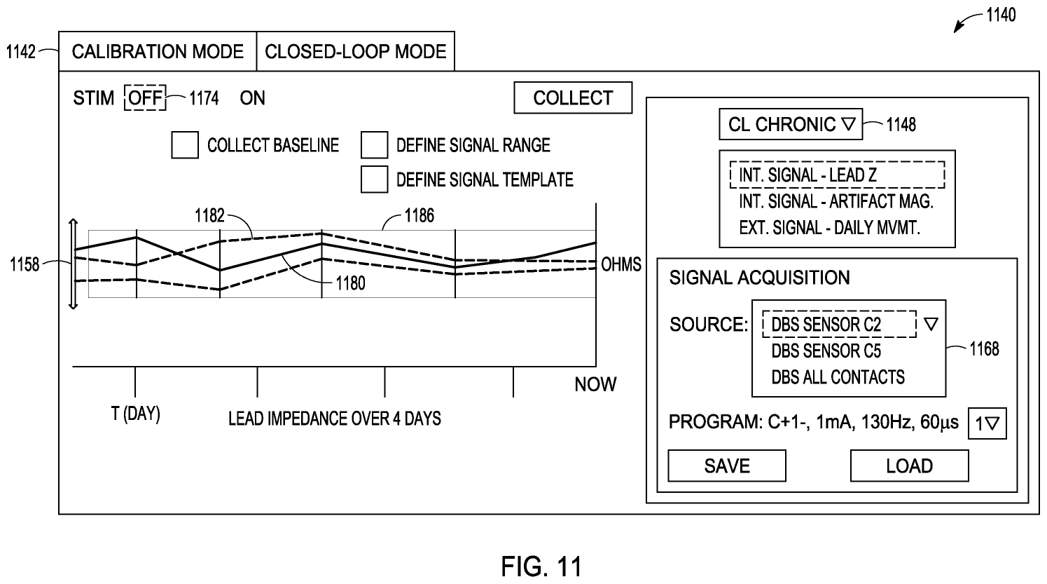

FIGS. 10A-10B are another example of a GUI screen useful for calibration of a neurostimulation device. The GUI screen 1040 is used to produce a signal template that can be used as the target response signal. In FIG. 10A, the highlighted tab 1042 indicates that the neurostimulation device is in calibration mode. A dropdown menu 1048 provides selection by the user of the type of signal template. In the example dropdown menu 1048, the stimulation evoked potential (STN EP) as sensed by the internal sensor (Int. Signal) is selected. The GUI screen 1040 includes a menu 1068 to select the signal source. In the example, the sensor selected is the DBS sensor which can include recording electrodes of the implanted lead or leads connected to one or more sense amplifiers of the IPG. The other option shown in the example is a sensor configuration that uses the case of the IPG as a recording electrode. The user can define the template when neurostimulation is on 1074 (STIM ON) or off (STIM OFF). The arrows 1058 indicate the signal range for the recording and waveform 1070 is the recorded signal. The window 1072 indicates the signal window for the target template. The user can drag and click the window location to define a signal template.

FIG. 10B shows an example of a result window 1076 for the template signal. The user is given the option to save the template, or the template can be discarded and another template can be produced. Templates may be pre-loaded as well.

In the examples of FIG. 10A-10B, the programming device (e.g., CP 18) of the neurostimulation device configures the target neural response signal in the neurostimulation device by sending a programming prompt to place the neurostimulation device in calibration mode to produce the target response signal which is stored in memory of the neurostimulation device.

In some examples, the programming prompt causes the neurostimulation device to send neural response information to the programmer (e.g., sampled values of a neural response signal). The programming device processes the response information to determine the target response signal and stores the target response signal in the neurostimulation device using a communication circuit such as by using a Bluetooth compatible circuit or other wireless communication circuit.

In some examples, an ETS determines the target response signal and the target response signal is sent to the neurostimulation device for closed loop operation. The ETS can include a neural signal sensing circuit that senses one or more neural response signals, and can include signal processing circuitry that produces the target neural response signal. The target neural response signal is transferred to the memory of the implantable neurostimulation device using a communication circuit such as by using the communication circuit.

FIG. 11 is another example of a GUI screen 1140 useful for calibration of a neurostimulation device. Other signals are more appropriate for chronic tracking and FIG. 11 is an example of a calibration GUI screen 1104 for chronic response tracking. The highlighted tab 1142 indicates that the neurostimulation device is in calibration mode. The GUI screen 1140 includes a “CL Chronic” dropdown menu 1148 that indicates that the closed loop (CL) calibration is being done using lead impedance of the patient. Other chronic signal options shown in the example dropdown menu 1148 include collecting an internal signal related to the simulation artifact magnitude and an external signal related to the cumulative daily movement of the patient. The GUI screen 1140 includes a menu 1168 to select the signal source. In the example, the sensor selected is a DBS sensor (DBS Sensor C2).

A change in therapy could trigger the acquisition of a chronic signal, and if it is beyond the user-defined or pre-loaded range, the device could send a flag or initiate reprogramming. Evaluation metrics such as signal range or variance configured during chronic tracking could be used to guide the closed-loop application of neurostimulation. A deviation in the chronic signal may indicate that a therapeutic change is needed and the closed loop operation needs recalibration.

The example GUI screen 1140 provides the option to select a signal range 1158 for the chronic signal. The “signal range” for a chronic signal is defined similarly as the range for an acute signal, but it may be acquired less frequently and may be only acquired under certain circumstances. In example, the lead impedance signal is acquired over 4 days. The lead impedance signal range could be specified across electrodes or for each individual electrode.

The signal graph shows the lead impedance signal waveform in Ohms for DBS sensor C2 1180 and DBS Sensor C5 1182. And the signal range arrow 1158 and signal box 1186 are for DBS sensor C2. The signal range arrow 1158 and signal box could be color coded to show for which signal the range is being configured.

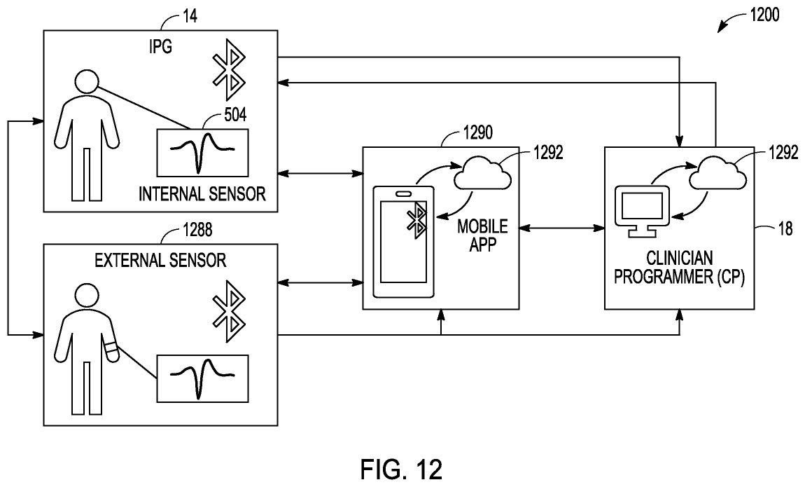

FIG. 12 is a block diagram of a medical device system. The system 1200 includes an IPG 14, a CP 18, an external sensor 1288, and a mobile device 1290. The external sensor 1288 detects a symptom of the neurological condition of the patient and can be any of the external sensors described herein. The mobile device 1290 can be a smartphone, tablet computer, or other mobile device. The mobile device 1290 includes a communication circuit to transfer information to a separate device, including the IPG 14, the CP 18 and the sensor 1288. In some examples, the sensor used to detect a symptom of the neurological condition of the patient is included in the mobile device 1290. The mobile device 1290 also includes a control circuit (e.g., one or more microprocessors) and a memory. The memory stores an application (or App). The App includes computer executable instructions that, when performed by the control circuit, cause the control circuit to perform the operations described.

The mobile device 1290 would be located with the patient and communicates with the CP 18 using a WiFi network (e.g., via the Internet), a cellular network, or a wireless personal area network (e.g., a Bluetooth link). In certain examples, one or more portions of the CP 18 are included in a cloud-based server. The mobile device 1290 performs two-way communication with the CP 18, and receives programming information from the CP 18 and transfers the programming information to the IPG 14.

The mobile device 1290 can transfer symptom information produced by the external sensor 1288 to the CP 18. The mobile device 1290 can receive neurostimulation parameters from the CP 18 and transfer the parameters to memory of the IPG 14. The mobile device 1290 can send a prompt to the IPG 14 to generate a target neural response signal, or the mobile device 1290 can send the target neural response signal to the IPG 14. In certain examples, the mobile device 1290 can prompt the IPG 14 to transmit samples of sensed neural response signals to the mobile device 1290 and the mobile device 1290 sends the sampled signal information to the CP 18 for signal processing to generate the target response signal.

The mobile device 1290 may also communicate a prompt received from the CP 18 to the IPG 14 to cause the neurostimulation device to enter closed loop operation, in which the IPG 14 recurrently adjusts the one or more neurostimulation parameters to reduce a difference between a neural response signal sensed by the neurostimulation device and the target neural response signal.

The mobile device 1290 may recurrently send symptom information to the CP 18 (e.g., according to a schedule). If the symptom indicates that the patient's condition has changed, the CP 18 may send a prompt to recalibrate the target to the mobile device 1290 that the mobile device 1290 relays to the IPG 14. In certain examples, if the symptom indicates that the patient's condition has changed, the CP 18 may send a new target response signal that the mobile device 1290 relays to the IPG 14.

In some examples, the App of the mobile device 1290 performs the functions of the CP 18. The App of the mobile device 1290 determines the neurostimulation parameters and sends the parameters to the IPG 14. The prompt to enter closed loop control operation originates from the App and is not relayed from a separate CP device. The App may perform the signal processing to generate the target response signal and store the target response signal in the IPG 14. The mobile device 1290 may include the external sensor 1288 and the App may generate the target response signal using information from the external sensor and the internal sensor of the IPG 14. The App may change the target response signal based on information from the external sensor.

In some examples, the mobile device communicates directly with the IPG 14. Physiological features can be extracted from sensed neural response signals and uploaded 24/7 to the cloud via an application running on the mobile device 1290. Of note, raw or minimally pre-processed segments of relevant physiological signals can be uploaded to the mobile device 1290 and from there to the CP 18 and the cloud 1292, or directly to the cloud 1292.

The several embodiments described herein provide device-based treatment for the neurological condition of the patient. Automatic therapy adjustments are made by the neurostimulation device without the need of additional clinician programming. The automatic adjustments are made by sensing the effect of the current neurostimulation in meeting or not meeting a treatment target. The neurostimulation device applies automatic closed loop feedback to adjust one or more parameters of the neurostimulation to bring the condition of the patient closer to the treatment target.

The embodiments described herein can be methods that are machine or computer-implemented at least in part. Some embodiments may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. Various embodiments are illustrated in the figures above. One or more features from one or more of these embodiments may be combined to form other embodiments.

The above detailed description is intended to be illustrative, and not restrictive. The scope of the disclosure should, therefore, be determined with references to the appended claims, along with the full scope of equivalents to which such claims are entitled.

What is claimed is:

1. A computer-implemented method of calibration of an implantable neurostimulation device, the method comprising:
sensing one or more symptoms of a neurological condition of a subject using one or more sensors external to the neurostimulation device;
delivering neurostimulation to the subject using the neurostimulation device and adjusting neurostimulation parameters based on the sensed one or more symptoms;
sensing one or more neural response signals resulting from the neurostimulation using a sensor of the neurostimulation device;
correlating the one or more sensed symptoms with the one or more sensed neural response signals;
determining a target neural response using the correlating; and
recurrently adjusting the neurostimulation parameters according to a comparison of subsequently sensed neural response signals to the target neural response signal.

2. The method of claim 1, including:
ending the adjusting of the neurostimulation parameters based on the one or more sensed symptoms; and
continuing the adjusting of the neurostimulation parameters according to the comparison of subsequently sensed neural response signals to the target neural response signal.

3. The method of claim 1, including:
detecting a change in at least one sensed symptom of the one or more sensed symptoms; and
changing the target neural response signal based on the detected change in the at least one sensed symptom.

4. The method of claim 1, including:
detecting a change in at least one sensed symptom of the one or more sensed symptoms; and
enabling the recurrent adjusting the neurostimulation parameters in response to the detected change in the at least one sensed symptom.

5. The method of claim 1, wherein the sensing the one or more neural response signals includes sensing one or more evoked potential signals, and the target neural response signal is a target evoked potential signal.

6. The method of claim 5, including sampling the one or more evoked potential signals; and
generating a template of the target evoked potential signal using one or more sampled evoked potential signals.

7. The method of claim 1, including:
sensing the one or more neural response signals using a device separate from the neurostimulation device;
generating the target neural response signal using the separate device; and
sending the target neural response signal to the neurostimulation device and storing the target neural response in memory of the neurostimulation device.

8. The method of claim 1, wherein the sensing the one or more neural response signals includes sensing one or more evoked resonant neural activity signals, one or more local field potential signals, or one or more stimulation artifact signals.

9. The method of claim 1, including:
sensing lead impedance of a lead used to deliver the neurostimulation;
compare the sensed lead impedance to a specified lead impedance range;
send an indication associated with the sensed lead impedance to a user or process when the sensed lead impedance is outside the specified lead impedance range.

10. The method of claim 1, including adjusting the neurostimulation parameters according to the sensing of the subsequent neural response signals and according to a medication schedule of the subject.

11. The method of claim 1, wherein the one or more sensed symptoms includes a tremor, and the one or more external sensors includes a motion sensor.

12. The method of claim 1, wherein the one or more sensed symptoms includes abnormal gait of the subject, and the one or more external sensors includes a motion sensor.

13. The method of claim 1, wherein the neurostimulation device is an implantable pulse generator that includes the internal sensor and the one or more external sensors are wearable sensors.

14. The method of claim 1, including recording one or more signals sensed by the external sensor in response to delivering the neurostimulation.

15. A medical device system, the system comprising:
an implantable neurostimulation device, including:
a therapy circuit configured to deliver electrical neurostimulation to a subject when coupled to implantable electrodes;
at least one external sensor configured to detect at least one symptom of a neurological condition of the subject;
an external device including:
an external control circuit configured to:
receive information of the at least one detected symptom from the external sensor, and set one or more neurostimulation parameters of the neurostimulation delivered by the neurostimulation device according to the at least one detected symptom; and
configure a target neural response signal in the neurostimulation device;
wherein the implantable neurostimulation device further includes:
a sensor circuit configured to sense one or more neural response signals resulting from the neurostimulation; and
an internal control circuit, operatively coupled to the therapy circuit and the sensor circuit, and configured to recurrently adjust the one or more neurostimulation parameters based on a comparison of the target neural response signal to the sensed neural response signals.

16. The system of claim 15, wherein the external device further includes:
a neural signal sensing circuit configured to sense the one or more neural signals;
signal processing circuitry configured to produce the target neural response signal; and
a communication circuit configured to transfer the target neural response signal to memory of the implantable neurostimulation device.

17. The system of claim 15, wherein the implantable neurostimulation device further includes:
a communication circuit configured to receive a prompt from the external device; and
signal processing circuitry configured to produce the target neural response signal in response to a prompt received from the external device.

18. An electronic device comprising:
one or more sensors configured to detect one or more symptoms of a neurological condition of a subject;
a communication circuit configured to transfer information to a separate device;
a control circuit operatively coupled to the one or more sensors and the communication circuit; and
a memory storing an application that includes instructions that when performed by the control circuit, causes the control circuit to perform operations including:
communicate one or more neurostimulation parameters of neurostimulation according to the detected one or more symptoms, wherein the neurostimulation is provided by a separate neurostimulation device;
initiate a transfer of a target neural response signal to the neurostimulation device; and
communicate a prompt to cause the neurostimulation device to recurrently adjust the one or more neurostimulation parameters to reduce a difference between a neural response signal sensed by the neurostimulation device and the target neural response signal.

19. The electronic device of claim 18, wherein the application includes instructions that when performed by the control circuit, causes the control circuit to perform operations including:
transfer symptom information of the detected symptom to a separate programming device; and
receive the target neural response signal from the separate device.

20. The electronic device of claim 18, wherein the one or more sensors, the communication circuit, the control circuit, and the memory are included in a mobile device.
